(19) United States
(12) Patent Application Publication
(10) Pub. No.: US 2022/0273664 A1
(43) Pub. Date: Sep. 1, 2022

(54) Mitochondrial Targeted Releasable Linker

(71) Applicant: THE GOVERNING COUNCIL OF THE UNIVERSITY OF TORONTO, Toronto (CA)

(72) Inventors: Shana KELLEY, Toronto (CA); Eric LEI, Etobicoke (CA)

(21) Appl. No.: 16/624,825

(22) PCT Filed: Jun. 21, 2018
(86) PCT No.: PCT/CA2018/000126

§ 371 Date: Dec. 19, 2019

Related U.S. Application Data
(60) Provisional application No. 62/524,161, filed on Jun. 23, 2017.

(51) Int. Cl. A61K 31/5377; A61K 47/64; A61K 47/54; A61K 31/4196; A61K 31/496; A61K 31/416; A61K 31/4468; A61K 31/415

(52) U.S. Cl. CPC A61K 31/5377; A61K 47/64; A61K 47/54; A61K 31/4196; A61K 31/496; A61K 31/416; A61K 31/4468; A61K 31/415

(57) ABSTRACT

There is described herein compound comprising a mitochondrial targeting portion, a cargo portion including a drug unit, and a linker conjugating the mitochondrial targeting portion and the cargo portion, the linker portion cleavable in a mitochondrion of a cell for preferentially releasing the cargo portion within the mitochondrion as compared to a cytoplasm of the cell.

### RELATED APPLICATION

This application claims priority to U.S. Provisional Application No. 62/524,161 filed on Jun. 23, 2017, which is incorporated herein by reference in its entirety.

### FIELD

This invention relates to compounds having cleavable linkers that preferentially deliver a drug to a mitochondrion of a cell.

### INTRODUCTION

The mitochondria of mammalian cells have a role in the production of energy and regulation of programmed cell death. They have a role in maintaining cellular health, and dysregulation of mitochondria has been connected with a variety of human diseases (see, for example, reference 1, listed below). The delivery of therapeutics or small molecules to this cellular organelle is challenging, however, because of the double-membrane structure of mitochondria that is difficult to penetrate (see, for example, reference 2, listed below).

A variety of molecular delivery systems that can transport cargo into mitochondria have been reported (see, for example, reference 2, listed below). For example, mitochondria penetrating peptides (MPPs), which are mitochondrial localization vectors, directly target small molecules to the mitochondrial matrix (see, for example, references 3-5, listed below). The use of MPPs for mitochondrial small molecule targeting has proven useful for the development of new probes for mitochondrial biology and investigating drug activities within the mitochondria with organellar specificity (see, for example, references 6-10, listed below). The use of MPPs for mitochondrial small molecule targeting was discussed in WO2011150494 filed May 27, 2011 and WO2011150493 filed May 27, 2011, both herein incorporated by reference in their entirety. However, the MPP conjugates generated and studied to date feature covalent and uncleavable linkers, and therefore the peptide remains attached to molecular cargo. While this approach has produced several interesting compounds with drug-like properties and significant levels of activity for a variety of probes, the presence of the delivery vehicle after transport to mitochondria is a limitation of MPPs and other mitochondrial delivery vectors.

A method for traceless release of a small molecule once it is trafficked to the mitochondrial matrix would benefit mitochondrial targeting vectors as a whole and expand the breadth of compounds that can be targeted in the organelle. Several existing examples of cargo release in the mitochondria have focused on taking advantage of enzymatic cleavage of a labile ester linker (see, for example, references 11-12, listed below). However, linkers that rely on enzymatic cleavage are particularly sensitive to sterics around the cleavage site. Small molecules with chemically tractable groups near bulkier substituents may inhibit access of cleavage enzymes, either limiting the breadth of compounds able to be conjugated or requiring chemical modification of the cargo for attachment. In addition, enzyme expression can vary by cell type, environment, and metabolic status which could make cleavage kinetics inconsistent.

There is a need for mitochondrial delivery of compounds with linkers cleavable by endogenous chemical agents that are suitable for mitochondrial small molecule targeting and release.

### SUMMARY

According to one aspect of the invention, there is provided a compound comprising: a mitochondrial targeting portion; a cargo portion including a drug unit; and a linker conjugating the mitochondrial targeting portion and the cargo portion, the linker portion cleavable in a mitochondrion of a cell for preferentially releasing the cargo portion within the mitochondrion as compared to a cytoplasm of the cell.

In another aspect, there is provided a compound having a structure according to Formula I:

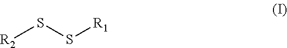

wherein R₁ is a mitochondrial targeting portion;
R₂ is a cargo portion including a drug unit; and
each carbon atom bonded to the disulfide is, independently, unsubstituted; mono- or di-substituted by, independently, a hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl group; or di-substituted such that the carbon atom bonded to the disulfide forms part of a C₃-C₈ cycloalkyl, or C₃-C₈ cycloalkenyl group.

In another aspect, there is provided the compound as described above for the treatment of cancer, a microbial infection, a neurodegenerative disorder, a metabolic disorder, or a mitochondrial disease.

In another aspect, there is provided a use of the compound as described above in the preparation of a medicament for the treatment of cancer, a microbial infection, a neurodegenerative disorder, a metabolic disorder, or a mitochondrial disease.

### BRIEF DESCRIPTION OF DRAWINGS

Embodiments of the invention may best be understood by referring to the following description and accompanying drawings. In the description and drawings, like numerals refer to like structures or processes.

FIG. 1 is a schematic representation of the delivery a drug into a mitochondrion using a compound according to an aspect of the present invention.

FIG. 2 is an overview of linkers tested for mitochondrial delivery and release of molecular cargo. Reporter conjugates contained the TAMRA fluorophore [Y] paired to a BHQ-2 quencher [X] through disulfide linkers. Compound 1 features an uncleavable linker, while compounds 2, 3, and 4 contained unsubstituted, mono-substituted, and di-substituted disulfides, respectively. Z represents the structure of a mitochondria-penetrating peptide.

FIG. 3A is a graphical representation of in vitro cleavage kinetics. Fluorescence recovery of the reporter conjugates in PBS incubated with 0.5 mM DTT.

FIG. 3B is a graphical representation of in cellulo cleavage kinetics. Fluorescence recovery of the indicated reporter conjugates in K562 cells. K562s were treated with the corresponding reporter then lysed. Lysates were split and the fluorescence of one sample was normalized to a second sample which was treated for 10 minutes with 25 mM TCEP as a fully cleaved control.

FIG. 4 illustrates the time-dependent fluorescence of reporter conjugates in living cells. A series of photographs show fluorescence microscopic images of cells treated with the reporter conjugates after 0.5 hours, 8 hours and 24 hours. Image acquisition settings were maintained between compounds and timepoints. The scale bar represents 20 μm.

FIG. 5 illustrates the localization of reporter conjugates in living cells. Peptide fluorescence corresponds to the green channel (the left column) and mitochondria labelled with the mitochondria-specific dye Mitotracker Deep Red is shown in the red channel (the middle column). Insets are outlined by the dashed boxes. The upper inset of each row is a zoomed in portion of the green channel and the lower inset of each row is a zoomed in portion of the red channel for the same reporter conjugate. Pearson's coefficients for Compound 2=0.95, Compound 3=0.76, Compound 4=0.77. The scale bar represents 20 μm.

FIG. 6A illustrates the chemical structure of the releasable Luminespib-MPP conjugate, compound 5.

FIG. 6B illustrates the chemical structure of the uncleavable Luminespib conjugate, compound 6.

FIG. 6C illustrates the chemical structure of parent drug, Luminespib.

FIG. 6D illustrates the chemical structure of a linker plus mitochondrial targeting vector.

FIG. 6E illustrates the chemical structure of the fluorescently labelled analogue of compound 5.

FIG. 6F illustrates the reaction mechanism of disulfide cleavage and auto-cyclization.

FIG. 6G illustrates the localization of a fluorescently labelled compound 5 with peptide fluorescence shown in the green channel (left image) and mitochondria labelled with Mitotracker Deep Red in the red channel (middle image). Insets are outlined in the dashed boxes with the upper inset being the zoomed in portion of the green channel and the lower inset being the zoomed in portion of the red channel. The Pearson's coefficient for this compound was 0.92. The scale bar represents 20 μm

FIG. 7A is a graphical representation of the toxicity of compound 5 at different time points in K562 cells.

FIG. 7B is a graphical representation of the toxicity of compound 6 at different time points in K562 cells.

FIG. 8A is a chart illustrating apoptosis as measured via Annexin V staining of K562 cells treated for 24 hours with 2.5 μM of the compounds indicated. Cell populations were gated with annexin V+/Sytox red− cells as early apoptotic, and annexin V+/Sytox red+ cells as late apoptotic. Necrotic cells, defined as Annexin V−/Sytox red+ cells were excluded as levels were negligible [<1%].

FIG. 8B is a chart illustrating the mitochondrial mass as measured by incubation with Mitotracker Green FM staining of K562 cells treated with 2.5 μM of the indicated compounds for 24 hours. Cells with Sytox red staining were gated against and excluded.

FIG. 8C is a chart illustrating mitochondrial membrane depolarization of K562 cells as measured by TMRM staining treated with 2.5 μM of the indicated compounds for 24 hours.

FIG. 9 is a chart illustrating the HPLC analysis of compound 5 pre cleavage (left plot) and post cleavage (right plot) with the reducing agent TCEP. A 50 μM stock solution of Compound 5 in PBS was run through RP-HPLC on a C18 column with a H₂O/MeCN gradient with 0.1% TFA and compared to a 50 μM stock solution of Compound 5 in PBS cleaved with 25 mM TCEP. Peak A corresponds to purified compound 5. Peak B was identified as Luminespib by comparison of retention time to an analytical standard and by mass spectrometry. Peak C was identified as the cleaved thiol-MPP fragment of compound 5 by comparison to an analytical standard and by mass spectrometry.

FIG. 10 is a graphical representation of the toxicity of luminespib (compound 7, as shown in FIG. 6C) at different time points in K562 cells.

FIG. 11 illustrates the chemical structure of the releasable aminocoumarin-MPP conjugate.

FIG. 12 is a chart illustrating the HPLC analysis of compound X pre cleavage (left plot) and post cleavage (right plot) with the reducing agent TCEP. A 50 μM stock solution of Compound 5 in PBS was run through RP-HPLC on a C18 column with a H₂O/MeCN gradient with 0.1% TFA and compared to a 50 μM stock solution of Compound X in PBS cleaved with 25 mM TCEP over 4 hours. Peak A corresponds to purified compound X. Peak B was identified as 7-amino-4-methylcoumarin by comparison of retention time to an analytical standard. Peak C was identified as the cleaved thiol-MPP fragment of compound 5 by comparison of retention time to an analytical standard.

FIG. 13 illustrates the localization of the releasable aminocoumarin-MPP, compound X, in living cells. Peptide fluorescence corresponds to the green channel (the left column) and mitochondria labelled with the mitochondria-specific dye Mitotracker Deep Red is shown in the red channel (the middle column). Insets are outlined by the dashed boxes. The upper inset is a zoomed in portion of the green channel and the lower inset of each row is a zoomed in portion of the red channel for the same reporter conjugate. Pearson's coefficient for Compound X=0.733. The scale bar represents 20 μm.

FIG. 14 illustrates the chemical structure of the releasable BIIB021-MPP conjugate.

FIG. 15 is a chart illustrating the HPLC analysis of compound X pre cleavage (left plot) and post cleavage (right plot) with the reducing agent TCEP. A 50 μM stock solution of Compound 5 in PBS was run through RP-HPLC on a C18 column with a H₂O/MeCN gradient with 0.1% TFA and compared to a 50 μM stock solution of Compound X in PBS cleaved with 25 mM TCEP over 4 hours. Peak A corresponds to purified compound X. Peak B was identified as BIIB021 (6-chloro-9-[(4-methoxy-3,5-dimethyl-2-pyridyl)methyl]-9H-purin-2-amine) by comparison of retention time to an analytical standard and by mass spectrometry. Peak C was identified as the cleaved thiol-MPP fragment of compound 5 by comparison to an analytical standard and by mass spectrometry.

### DETAILED DESCRIPTION

In the following description, numerous specific details are set forth to provide a thorough understanding of the invention. However, it is understood that the invention may be practiced without these specific details.

“Small molecule” means an organic compound that may help regulate a biological process and having a low molecular weight. In some embodiments, the small molecule has molecular weight of less than 900 daltons, or even less than 500 daltons.

“Peptide” means an oligomer comprising amino acid monomers linked by peptide bonds. In some embodiments, the peptide has from 3 amino acids to 30 amino acids.

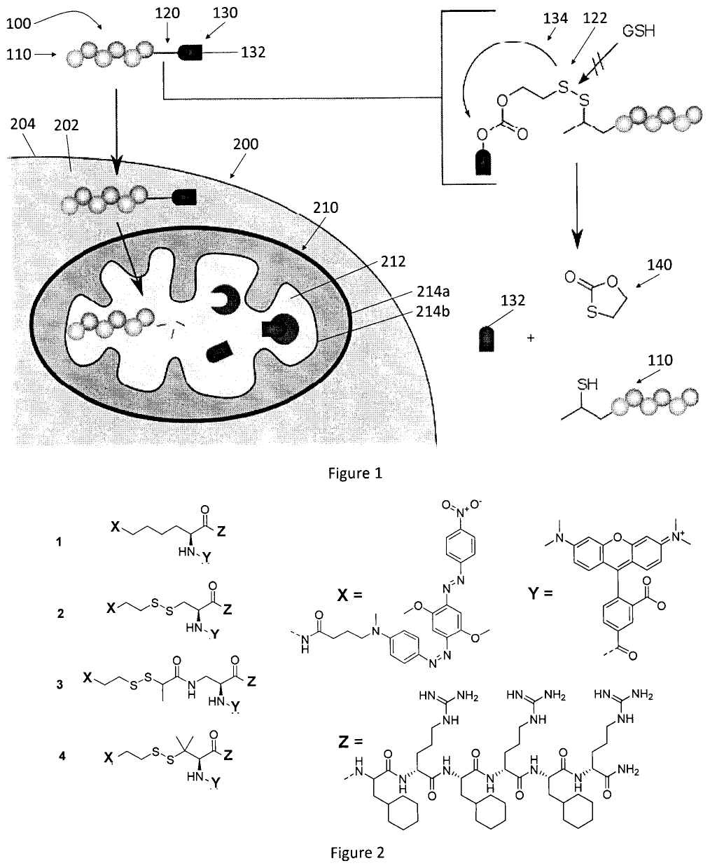

Having reference to FIG. 1, in an aspect of the invention, there is provided a compound 100 comprising a mitochondrial targeting portion 110, a cargo portion 130 including a drug unit 132, and a linker 120 conjugating the mitochondrial targeting portion 110 and the cargo portion 130, the linker 120 cleavable in a mitochondrion 210 of a cell 200 for preferentially releasing the cargo portion 130 within mitochondria 210 as compared to a cytoplasm 202 of the cell 200.

The mitochondrial targeting portion 110 facilitates the transport of the compound 100 across both the plasma membrane 204 and the mitochondrial membranes 214a,b. In some embodiments, the mitochondrial targeting portion 110 includes a mitochondrial penetrating peptide (MPP), a triphenylphosphonium (TPP), a transactivator of transcription peptide fused mitochondrial targeting sequence (TAT-MTS), a mitochondrial protein, or a small molecule with mitochondrial localization.

In some embodiments, the mitochondrial targeting portion 110 includes an MPP. In some embodiments, the MPP contains cationic and hydrophobic residues to provide a positively charged lipophilic character that facilitates passage through both the plasma membrane 204 and mitochondrial membranes 214a,b. In some embodiments, the MPP is both lipophilic and cationic. In some embodiments, the cationic residues include lysine (K), arginine (R), aminophenylalanine, ornithine, or a combination thereof, to provide positive charge. In some embodiments, the hydrophobic residues include phenylalanine (F), cyclohexylalanine (Fₓ), 2-aminooctanoic acid (Hex), diphenylalanine (DIF), (1-naphthyl)-L-alanine (Nap) or any combination thereof to impart lipophilicity. Although the arrangement of charged and hydrophobic residues within an MPP is not particularly restricted, provided the MPP possesses appropriate charge and lipophilicity to pass through the plasma membrane 204 and the mitochondrial membranes 214a,b, the MPPs may comprise alternating cationic and hydrophobic residues to increase the level of lipophilicity within the MPP.

In some embodiments, the MPP crosses the membrane in a potential dependent manner. In some embodiments, the MPP comprises amino acid residues modified to provide intracellular stability. Such residues include, for example, d-stereoisomers, an amide terminus or both. In some embodiments, the MPP comprises a charge of +3 and a log P value of at least about −1.7. In some embodiments, the MPP comprises a charge of +5 and a log P value of at least about −2.5.

Considerations and discussion regarding the design of MPPs can be found, for example, in Sae Rin Jean et al, “Peptide-Mediated Delivery of Chemical Probes and Therapeutics to Mitochondria”, (2016) 49 Acc Chem Res 1893; Sae Rin Jean et al, “Molecular Vehicles for Mitochondrial Chemical Biology and Drug Delivery”, (2014) 9, ACS Chem Biol 323; and Kristin L Horton et al, “Mitochondria-Penetrating Peptides”, (2008) 15 Chem Biol 375; which are hereby incorporated by reference in their entirety.

In some embodiments, the MPP comprises an amino acid sequence set out in Table 1, below.

TABLE 1

| SEQ ID NO. | Compound |
|---|---|
| 1 | Fₓ—r-Fₓ—K |
| 2 | Fₓ—r-Fₓ—K—Fₓ—r-F—K |
| 3 | F—r-Fₓ—K—F—r-Fₓ—K |
| 4 | F—r-Fₓ—K |
| 5 | F—r-DIF-K |
| 6 | F—r-Nap-K |
| 7 | F—r-HEX-K |
| 8 | (Fₓ-r)₄ |
| 9 | (Fₓ-r-G-r)₃ |
| 10 | Fₓ—r-Fₓ—r-Fₓ—r |

r = D-arginine
Fₓ = cyclohexylalanine
Nap = (1-naphthyl)-L-alanine
Hex = 2-aminooctanoic acid
DIF = diphenylalanine
K = lysine
G = glycine
F = phenylalanine

In some embodiments, the MPP comprises the sequence set forth in SEQ ID NO: 10.

Other suitable MPPs may be found, for example, in Kristin L Horton et al, “Mitochondria-Penetrating Peptides” (2008) 15 Chem Biol 375; and Kristin L Horton et al, “Tuning the Activity of Mitochondria-Penetrating Peptides for Delivery or Disruption”, (2012) 13 ChemBioChem 476, which are hereby incorporated by reference in their entirety.

In some embodiments, the linker 120 is preferentially cleaved in the cell 200 as compared to an extra-cellular region. In some embodiments, the linker 120 is cleaved after the compound 100 is transported into a matrix 212 of a mitochondrion 210. In some embodiments, a cleavage agent in the matrix 212 of the mitochondrion 210 cleaves the linker 120. In some embodiments, the cleavage agent is present in both the cytoplasm 202 and the mitochondrial matrix 212 of the cell 200 and the compound 100 is preferentially cleaved in the mitochondrion 210. As such, in some embodiments, the compound 100 is designed to mitigate against the premature cleavage of the linker 120 in the cytoplasm 202. For example, the ratio of the rate of cleavage of the compound in the cytoplasm 202 to the rate of cleavage of the compound in the mitochondria 210 may be affected by steric effects. In some embodiments, from 2 to 6% of the total molecules of the compound are cleaved before the compound is localized to the mitochondria. Without wishing to be bound by theory, it is believed that steric effects at the linker interferes with the ability of a cleavage agent from cleaving the linker, thereby providing an opportunity for the mitochondrial targeting portion to facilitate the transport of the compound to a mitochondrion.

The cargo portion 130 and the mitochondrial targeting portion 110 may contribute to steric effects at the linker 120. Steric effects at the linker 120 may be modified by introducing one or more substitutions at or near the linker. Generally, the degree of substitution decreases the rate at which the compound is cleaved. For example, in compounds having the same mitochondrial targeting portions and cargo portions, one with an unsubstituted linker may be cleaved at a faster rate than one with a mono-substituted linker, which may be cleaved at a faster rate than one with a di-substituted linker. The release profile of drug unit into the mitochondria may be modulated depending on the desired use.

In some embodiments, the linker is a hydrolysis sensitive linker or disulfide linker. In some embodiments, the linker 120 includes a disulfide bond. In some embodiments, the disulfide bonds are cleavable by cellular thiols, cellular antioxidants, reducing agents, or any combination thereof. Exemplary reducing agents for cleaving the linker 120 include glutathione. Glutathione is present endogenously in cells 200, including in the cytoplasm 202 and in the mitochondrial matrix 212, but is relatively scarce in the extra-cellular environment. Since the relative concentrations of glutathione in the cytoplasm 202 and mitochondrial matrix 212 in a cell 200 may be similar, the linker 120 resists cleavage by glutathione until the compound 100 can be transported into the mitochondrial matrix 212.

In some embodiments where the linker 120 includes a disulfide moiety, each carbon atom bonded to the disulfide is, independently, un-substituted; mono- or di-substituted by, independently, a hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl group; or di-substituted such that the carbon atom bonded to the disulfide forms part of a C₃-C₈ cycloalkyl, C₃-C₈ cycloalkenyl group.

In some embodiments, the cleavage of the linker 120 releases the cargo portion 130. The cargo portion 130 includes a drug unit 132. In some embodiments, the drug unit 132 includes or is functionalized with a hydroxyl, amino, or thiol moiety.

In some embodiments, the cargo portion 130 is the drug unit 132, and includes a portion of the cleaved linker 120. In some embodiments, the linker 120 is a disulfide and cleavage occurs at the sulfur-sulfur bond such that one of the sulfurs of the disulfide forms a thiol moiety of the drug unit 132.

In some embodiments, the cargo portion 130 includes an auto-cyclization moiety 134 that activates by the cleavage of the linker 120 to release the drug unit 132. In some embodiments, the auto-cyclization moiety 134 reacts with a moiety of the cleaved linker portion to effect the release of the drug unit 132. In some embodiments, the auto-cyclization moiety 134 includes the moiety 122 of the cleaved linker portion to effect the release of the drug unit 132. In some embodiments, the auto-cyclization moiety 134 includes a drug-joining moiety. In some embodiments, the drug unit 132 includes a cargo-joining moiety or is functionalized with a cargo-joining moiety bonded to the auto-cyclization moiety 134, for example, at the drug-joining moiety to form the cargo portion 130. In some embodiments, the bond between the cargo-joining moiety and the drug-joining moiety is cleaved by the moiety 122 of the cleaved linker portion.

In some embodiments, the drug-joining moiety includes an ester moiety. In some embodiments, the linker 120 is a disulfide and cleavage occurs at the sulfur-sulfur bond such that the moiety of the cleaved linker portion that cleaves the bond between the cargo-joining moiety and the drug-joining moiety includes a thiol moiety. The thiol moiety cleaves the bond between the cargo-joining moiety and the drug-joining moiety, thereby cleaving the cargo portion 130 and releasing the drug unit 132. For example, in some embodiments, the drug-joining moiety includes an ester moiety and the thiol moiety cleaves the bond between the cargo-joining moiety and the ester such that the drug unit is released and the ester moiety and the thiol moiety bond to form a cyclic monothiocarbonate 140.

In some embodiments, the cargo-joining moiety is an oxygen or nitrogen moiety bonded to the drug-joining moiety. In some embodiments, the drug-joining moiety and the cargo-joining moiety are bonded such that they form a carbonate or carbamate moiety. The cleavage of the drug-joining moiety and the cargo-joining moiety release the drug unit, which includes a hydroxyl or amino group, or is functionalized with a hydroxyl or amino group.

In some embodiments, the cargo-joining moiety of the drug unit contributes to the drug's activity. For compounds including such drug units, if the drug unit 132 is not released from conjugation, the effect of the drug unit 132 would be limited. For example, for the HSP90 inhibitor luminespib, functional groups involved in protein binding may also be necessary for their conjugation to the mitochondrial targeting portion.

In some embodiments, the drug unit 132 is for the treatment of a disorder that is associated with the mitochondria. For example, the disorder may be cancer, a microbial infection, a neurodegenerative disorder, a metabolic disorder, or a mitochondrial disease.

In some embodiments, the drug unit 132 is a small molecule drug or a peptide. The use of relatively small cargo units is preferred over larger macromolecule units because large macromolecule units may result in the decrease the rate at which the compound is transported into the mitochondrial matrix. This may prevent translocation of the compound to the inner mitochondrial matrix. Further, larger macromolecule units are more difficult to conjugate with the linker and mitochondrial targeting portion.

In some embodiments, the drug unit is drug that is preferentially delivered to the mitochondria of a cell. For example, in some embodiments, the drug unit is a heat shock protein p90 (HSP90) inhibitor, pyruvate dehydrogenase kinase modulator, SIRT1 modulator, mitochondrial estrogen receptor ligand, mtDNA synthesis modulator, modulator of mtDNA fidelity, mitochondrial pol theta modulator, cyclophilin modulator, mitochondrial metabolism modulator, hexokinase modulator, lactate dehydrogenase modulator, glucose-6-phosphate modulator, kynurenine 3-monooxygenease modulator, AMP-activated protein kinase modulator, POLRMT modulator, or PINK1 modulator.

In some embodiments, the drug unit is an HSP90 inhibitor. In some embodiments, the HSP90 inhibitor includes luminespib, ganetespib, onalespib, SNX-2112, SNX-5422, KW2478, NMS-E973, VER-49009, or VER-50589. In some embodiments, the HSP90 inhibitor is luminespib.

In an aspect of the invention, there is provided a compound having a structure according to Formula I:

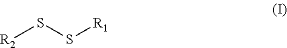

where R₁ is a mitochondrial targeting portion; R₂ is a cargo portion including a drug unit; and each carbon atom bonded to the disulfide is, independently, unsubstituted; mono- or di-substituted by, independently, a hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl group; or di-substituted such that the carbon atom bonded to the disulfide forms part of a C₃-C₈ cycloalkyl, or C₃-C₈ cycloalkenyl group.

In some embodiments, the mitochondrial targeting portion includes a mitochondrial penetrating peptide (MPP), a triphenylphosphonium (TPP), a transactivator of transcription peptide fused mitochondrial targeting sequence (TAT-MTS), a mitochondrial protein, or a small molecule with mitochondrial localization.

In some embodiments, R₁ has a structure according to Formula II:

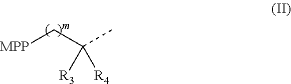

wherein R₃ and R₄ are, independently, hydrogen, hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl; or R₃ and R₄ together form C₃-C₈ cycloalkyl, or C₃-C₈ cycloalkenyl; and m is an integer from 0 to 8.

In some embodiments, the MPP has a structure according to Formula IIa:

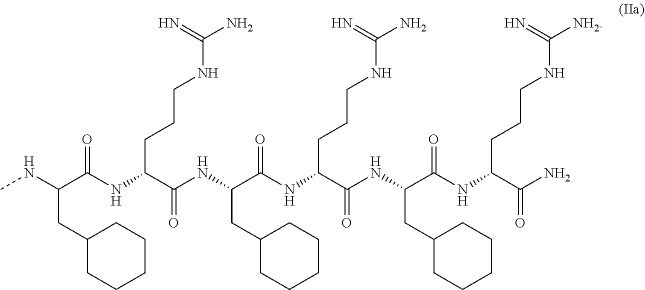

In some embodiments, R₂ has a structure according to Formula III:

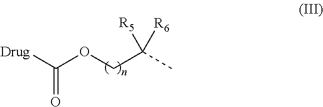

where R₅ and R₆ are, independently, hydrogen, hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl; or R₅ and R₆ together form C₃-C₈ cycloalkyl, or C₃-C₈ cycloalkenyl; n is an integer from 1 to 4; and Drug is the drug unit.

According to a further aspect, there is provided a pharmaceutical composition comprising the compound described herein and a pharmaceutically acceptable carrier. As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

According to a further aspect, there is provided the compound described herein for use in the treatment of cancer, a microbial infection, a neurodegenerative disorder, a metabolic disorder, or a mitochondrial disease.

According to a further aspect, there is provided the compound described herein for use in the preparation of a medicament for the treatment of cancer, a microbial infection, a neurodegenerative disorder, a metabolic disorder, or a mitochondrial disease.

The following examples are illustrative of various aspects of the invention, and do not limit the broad aspects of the invention as disclosed herein.

### EXAMPLES

### Materials and Methods

Disulfides were used as a basis for a releasable linker due to the presence of reducing agents, particularly glutathione, in the cell while being relatively scarce in the external environment (see, e.g. reference 13, listed below). These properties have been used successfully by a number of peptide based cytosolic delivery agents (see, e.g. references 14, 15, listed below). As relative concentrations of glutathione in the mitochondrial matrix and the cytoplasm are similar (see, e.g. reference 16, listed below), the stability of a disulfide-based linker as it passes through the cytoplasm was tested and optimized. A reporter system for linker stability (FIG. 2) was developed by combining a MPP (Z)-conjugated fluorophore (Y) with a fluorescence quencher (X) linked by a disulfide bond. Proximity-based quenching of the fluorescence occurs when the linker is intact, but is disrupted upon disulfide cleavage, leading to a fluorescence turn-on signal.

### General Peptide Synthesis

Solid phase peptide synthesis was performed on Rink amide MBHA resin (Novabiochem, UK) using a Prelude Protein Technologies peptide synthesizer as described previously [1]. Fx=L-cyclohexylalanine, r=N^{ω}-(2,2,4,6,7-pentamethyldihydrobenzofuran-5-sulfonyl)-D-arginine, K(Mtt)=N-ε-4-methyltrityl-L-lysine.

### Synthesis of S-trityl-2-mercaptoproprionic acid

10 mmol 2-mercaptoproprionic acid (Sigma-Aldrich, St. Louis Mo.) was dissolved in 5 mL dichloromethane (DCM) with trityl-chloride (1.1 eq, Sigma-Aldrich). The reaction was mixed for 72 hours, dried, and purified using RP-HPLC on a C18 column with an Acetonitrile/H₂O gradient with 0.1% TFA. The compound was identified by DART mass spectrometry, expected m/z=347.11, found m/z=347.1.

### Synthesis of Compound 1

25 μmol of NH₂-Fx-r-Fx-r-Fx-r on resin was reacted with N-α-Fmoc-N-ε-4-methyltrityl-L-lysine (4 eq, ChemPep Inc.), O-(benzotriazol-l-yl)-N,N,N′,N′-tetramethyl-uronium hexafluorophosphate (HBTU, 4 eq, Protein Technologies, Tucson Ariz.), and N,N-diisopropylethylamine (DIPEA, 8 eq, Sigma-Aldrich) in 1 mL N,N- dimethyl formamide (DMF) for 2 hours at room temperature. The resin was washed twice with DMF, methanol (MeOH), and DCM and deprotected using trifluoroacetic acid:triisopropylsilane:DCM (3:3:94, 2×15 minutes). The beads were washed then reacted with BHQ-2 carboxylic acid (2 eq, BioSearch Technologies, Petaluma Calif.), PyBOP (2 eq, ChemPep Inc.), and DIPEA (4 eq) in 1 mL DMF overnight. The peptide was washed and deprotected twice with 1 mL 20% piperidine in DMF (Protein Technologies) for 20 minutes. The peptide was lyophilized and reacted with 5-Carboxytetramethylrhodamine (2 eq, Anaspec, Freemont, Calif.), HBTU (2 eq), and DIPEA (4 eq) in 0.5 mL DMF for 2 hours. The peptide was cleaved from resin using trifluoroacetic acid:triisopropylsilane:water (95:2.5:2.5) and precipitated in ether at −20° C. for 1 hour. The peptide was purified by HPLC, then lyophilized. The peptide was identified by ESI mass spectrometry, expected m/z=1973.09, found m/z=1973.10.

### Synthesis of Compound 2

25 μmol of NH₂-Fx-r-Fx-r-Fx-r on resin was reacted with N-α-Fmoc-S-trityl-L-cysteine (4 eq, ChemPep Inc., Wellington Fla.), HBTU (4 eq, Protein Technologies, Tucson Ariz.), and DIPEA (8 eq) in 1 mL N,N- dimethyl formamide (DMF) for 2 hours at room temperature. The resin was washed twice with DMF, methanol (MeOH), and DCM and deprotected using trifluoroacetic acid:triisopropylsilane:DCM (3:3:94, 2×15 minutes). The beads were then equilibrated in acetonitrile:water (5:1) for 5 minutes, and cysteamine (20 eq, Sigma-Aldrich) in 1 mL acetonitrile:water (5:1) was added under mixing followed by iodine (10 eq, Sigma-Aldrich). The reaction was stirred vigorously for 30 minutes, followed washing (2×DMF/MeOH/DCM). The beads were then reacted with BHQ-2 carboxylic acid (2 eq), PyBOP (2 eq) and DIPEA (4 eq) in 1 mL DMF overnight. The peptide was washed (2×DMF/MeOH/DCM) and deprotected twice with 1 mL 20% piperidine in DMF for 20 minutes. The peptide was cleaved from resin using trifluoroacetic acid:triisopropylsilane:water (95:2.5:2.5) and precipitated in ether at −20° C. for 1 hour. The peptide was purified using RP-HPLC on a C18 column with a MeCN/H₂O gradient with 0.1% TFA. The peptide was lyophilized and reacted with 5-Carboxytetramethylrhodamine (2 eq), HBTU (2 eq), and DIPEA (4 eq) in 0.5 mL DMF for 2 hours. The peptide was re-precipitated in ether at −20° C. for 1 hour and then purified using RP-HPLC. The peptide was identified by ESI mass spectrometry, expected m/z=2024.03, found m/z=2024.03.

### Synthesis of Compound 3

50 μmol of NH₂-Fx-r-Fx-r-Fx-r on resin was reacted with Fmoc-N^{β}-Boc-L-2,3-diaminopropionic acid (4 eq, ChemPep Inc.), HBTU (4 eq), and DIPEA (8 eq) in 1 mL N,N-dimethyl formamide (DMF) for 2 hours at room temperature. The peptide was washed (2×DMF/MeOH/DCM), cleaved from resin using trifluoroacetic acid:triisopropylsilane:water (95:2.5:2.5) and precipitated in ether at −20° C. for 1 hour. The precipitate was purified by RP-HPLC, lyophilized, and reacted with S-trityl-2-mercaptoproprionic acid (4 eq), PyBOP (4 eq), and DIPEA (8 eq) in 0.5 mL DMF. The peptide was re-precipitated in ether and then dried under vacuum for 1 hour. The peptide was deprotected using 0.5 mL trifluoroacetic acid:triisopropylsilane:DCM (5:3:92, 15 minutes), precipitated in ether, and purified by HPLC. The peptide was dried under vacuum and dissolved in 0.5 mL acetonitrile:water (5:1). Cysteamine (20 eq) was added to the reaction mixture followed by iodine (10 eq) and the reaction was stirred for 30 minutes. The reaction mixture was precipitated in ether, and purified by HPLC. The peptide was lyophilized and reacted with BHQ-2 carboxylic acid (2 eq), PyBOP (2 eq), and DIPEA (4 eq) overnight in 0.5 mL DMF. The peptide was precipitated in ether, dried, and deprotected in 1 mL 20% piperidine in DMF for 20 minutes. The peptide was purified by HPLC, lyophilized, and reacted with 5-Carboxytetramethylrhodamine (2 eq), HBTU (2 eq), and DIPEA (4 eq) in 0.5 mL DMF for 2 hours. The peptide was precipitated in ether and purified by HPLC. The peptide was identified by ESI mass spectrometry, expected m/z=2094.07, found m/z=2094.07.

### Synthesis of Compound 4

25 μmol of NH₂-Fx-r-Fx-r-Fx-r on resin was reacted with Fmoc-S-trityl-L-penicillamine (4 eq, ChemPep Inc.), HBTU (4 eq), and DIPEA (8 eq) in 1 mL N,N-dimethyl formamide (DMF) for 2 hours at room temperature. The peptide was then reacted identically as Compound 2. The peptide was identified by ESI mass spectrometry, expected m/z=2051.06, found m/z=2051.06.

### Synthesis of Compound 5

25 μmol of NH₂-Fx-r-Fx-r-Fx-r on resin was reacted with S-trityl-2-mercaptoproprionic acid (4 eq), PyBOP (4 eq), and DIPEA (8 eq) in 1 mL DMF. The peptide was washed (2×DMF/MeOH/DCM), cleaved from resin using trifluoroacetic acid:triisopropylsilane:water (95:2.5:2.5) and precipitated in ether at −20° C. for 1 hour. The precipitate was purified by RP-HPLC, dried under vacuum and dissolved in 0.5 mL acetonitrile:water (5:1). 2-mercpatoethanol (20 eq, Sigma-Aldrich) was added to the reaction mixture followed by iodine (10 eq) and the reaction was stirred for 30 minutes. The peptide was purified by HPLC and lyophilized. 5-(2,4-Dihydroxy-5-isopropylphenyl)-N-ethyl-4-(4-(morpholinomethyl)phenyl)isoxazole-3-carboxamide (Luminespib, 3 eq, Adooq Bioscience, Irvine Calif.) was reacted with N,N′-Disuccinimidyl carbonate (3 eq, Sigma-Aldrich) and 4-(Dimethylamino)pyridine (12 eq, Sigma-Aldrich) in 0.4 mL DMF for 1 hour. The peptide was dissolved in 0.1 mL DMF and added to the reaction mixture and the solution was left stirring overnight. The peptide was precipitated in ether and purified by HPLC. Two isomers were identified during HPLC purification, likely due to attachment to either of the two resorcinol hydroxyls. The earlier eluting isomer was purified and tested due to its higher relative abundance. The solution was frozen in dry ice as the compound eluted from the column and lyophilized. The peptide was identified by ESI mass spectrometry, expected m/z=1599.88, found m/z=1599.88. The peptide was quantified via absorbance spectrophotometry using a SpectraMax M5 spectrophotometer. The absorbance profile of Compound 5 was found to be shifted as compared to Luminespib itself, therefore the peptide was quantified by cleavage in 25 mM TCEP in PBS pH 7.4 for 10 minutes, then measuring free Luminespib absorbance at 305 nm with an extinction coefficient of 8520 M⁻¹ cm⁻¹. TCEP was not found to affect the extinction coefficient of Luminespib.

### Synthesis of Compound 6

25 μmol of NH₂-Fx-r-Fx-r-Fx-r on resin was reacted with 3-[2-(2-Bromoethoxy)ethoxy]propanoic acid (4 eq, BroadPharm, San Diego Calif.), HBTU (4 eq), and DIPEA (8 eq) in 1 mL DMF. The peptide was washed (2×DMF/MeOH/DCM), cleaved from resin using trifluoroacetic acid:triisopropylsilane:water (95:2.5:2.5) and precipitated in ether at −20° C. for 1 hour. The precipitate was purified by RP-HPLC, lyophilized, and dissolved in 1 mL DMF. Luminespib (2 eq), and solid potassium carbonate (10 eq, Sigma-Aldrich) was added to the reaction mixture. The suspension was stirred overnight, filtered, then precipitated in ether and purified by HPLC. Two isomers were identified during HPLC purification, likely due to attachment to either of the two resorcinol hydroxyls. The earlier eluting isomer was purified and tested due to its higher relative abundance. The peptide was identified by ESI mass spectrometry, expected m/z=1551.97, found m/z=1551.97. The absorbance profile of Compound 6 was not found to be shifted as compared to Luminespib itself, therefore the peptide was quantified by measuring Luminespib absorbance at 305 nm with an extinction coefficient of 8520 M⁻¹ cm⁻¹.

### Synthesis of Fluorescently Labelled Compound 5

25 μmol of NH₂-Fx-r-Fx-r-Fx-r-K(Mtt) on resin was reacted with S-trityl-2-mercaptoproprionic acid (4 eq), PyBOP (4 eq), and DIPEA (8 eq) in 1 mL DMF. The peptide was washed (2×DMF/MeOH/DCM) and deprotected with trifluoroacetic acid:triisopropylsilane:DCM (5:3:92, 2×15 minutes). The peptide washed and equilibrated in acetonitrile:water (5:1). Cysteamine (20 eq) was dissolved in 1 mL acetonitrile:water (5:1) and added to the reaction mixture followed by iodine (10 eq). The reaction was stirred for 30 minutes. The peptide was washed (2×DMF:MeOH:DCM) and reacted with 5-Carboxytetramethylrhodamine (2 eq), HBTU (2 eq), and DIPEA (4 eq) in 0.5 mL DMF for 2 hours. The peptide was washed, cleaved from resin using trifluoroacetic acid:triisopropylsilane:water (95:2.5:2.5) and precipitated in ether at −20° C. for 1 hour. The precipitate was purified by HPLC and lyophilized. 5-(2,4-Dihydroxy-5-isopropylphenyl)-N-ethyl-4-(4-(morpholinomethyl)phenyl)isoxazole-3-carboxamide (Luminespib, 3 eq, Adooq Bioscience, Irvine Calif.) was reacted with N,N′-Disuccinimidyl carbonate (3 eq, Sigma-Aldrich) and 4-(Dimethylamino)pyridine (12 eq, Sigma-Aldrich) in 0.4 mL DMF for 1 hour. The peptide was dissolved in 0.1 mL DMF and added to the reaction mixture and the solution was left stirring overnight. The peptide was precipitated in ether and purified by HPLC. The earlier eluting isomer was purified and tested due to its higher relative abundance. The solution was frozen in dry ice as the compound eluted from the column and lyophilized. The peptide was identified by ESI mass spectrometry, expected m/z=2140.12, found m/z=2140.12. The peptide was quantified using the 5-Carboxytetramethylrhodamine absorbance at 547 nm with an extinction coefficient of 92000 M⁻¹ cm⁻¹.

### Synthesis of Aminocoumarin Peptide

25 μmol of NH₂-Fx-r-Fx-r-Fx-r on resin was reacted with S-trityl-2-mercaptoproprionic acid (4 eq), PyBOP (4 eq), and DIPEA (8 eq) in 1 mL DMF. The peptide was washed (2×DMF/MeOH/DCM), cleaved from resin using trifluoroacetic acid:triisopropylsilane:water (95:2.5:2.5) and precipitated in ether at −20° C. for 1 hour. The precipitate was purified by RP-HPLC, dried under vacuum and dissolved in 0.5 mL acetonitrile:water (5:1). 2-mercpatoethanol (20 eq, Sigma-Aldrich) was added to the reaction mixture followed by iodine (10 eq) and the reaction was stirred for 30 minutes. The peptide was purified by HPLC and lyophilized. 7-amino-4-methylcoumarin (3 eq, Sigma-Aldrich) was reacted with N,N′-Disuccinimidyl carbonate (3 eq, Sigma-Aldrich) and 4-(Dimethylamino)pyridine (6 eq, Sigma-Aldrich) in 0.4 mL DMF for 2 hours. The peptide was dissolved in 0.1 mL DMF along with 4-(Dimethylamino)pyridine (6 eq) and added to the reaction mixture and the solution was left stirring overnight. The compound was purified via HPLC and lyophilized. The peptide was identified by ESI mass spectrometry, expected m/z=1309.72, found m/z=1309.72. The peptide was quantified using the 7-amino-4-methylcoumain absorbance at 340 nm with an extinction coefficient of 10389 M⁻¹ cm⁻¹ in methanol.

### Synthesis of BIIB021 Peptide

25 μmol of NH₂-Fx-r-Fx-r-Fx-r on resin was reacted with Fmoc-S-trityl-L-penicillamine (4 eq, ChemPep Inc.), HBTU (4 eq), and DIPEA (8 eq) in 1 mL N,N-dimethyl formamide (DMF) for 2 hours at room temperature. The peptide was washed (2×DMF/MeOH/DCM), cleaved from resin using trifluoroacetic acid:triisopropylsilane:water (95:2.5:2.5) and precipitated in ether at −20° C. for 1 hour. The precipitate was purified by RP-HPLC, dried under vacuum and dissolved in 0.5 mL acetonitrile:water (5:1). 2-mercpatoethanol (20 eq, Sigma-Aldrich) was added to the reaction mixture followed by iodine (10 eq) and the reaction was stirred for 30 minutes. The peptide was purified by HPLC and lyophilized. BIIB021 ((6-chloro-9-[(4-methoxy-3,5-dimethyl-2-pyridyl)methyl]-9H-purin-2-amine), 2 eq, Selleck Chemicals, Houston Tex.) was dissolved in 0.2 mL DCM along with 4-(Dimethylamino)pyridine (4 eq). The solution was chilled to −10° C., and Triphosgene (0.71 eq, Sigma-Aldrich) was added and stirred for 10 minutes. The peptide was dissolved in 0.2 mL DMF and added to the solution, followed by stirring overnight at room temperature. The compound was purified via HPLC and lyophilized. The peptide was identified by ESI mass spectrometry, expected m/z=1537.80, found m/z=1537.82. The peptide was quantified using the BIIB021 absorbance at 310 nm with an extinction coefficient of 8295 M⁻¹ cm⁻¹ in PBS following overnight cleavage by 50 mM TCEP as the characteristic BIIB021 absorbance was found to shift while attached to the peptide.

### Example 1

Three linkers featuring thiols with differing levels of substitution were tested to determine their effect on the modulation of intracellular stability to identify which structure would maximize delivery of small molecule cargo. Three reporter conjugates featuring the different linkages (Compounds 2-4, FIG. 2) were synthesized and compared against an uncleavable control (Compound 1).

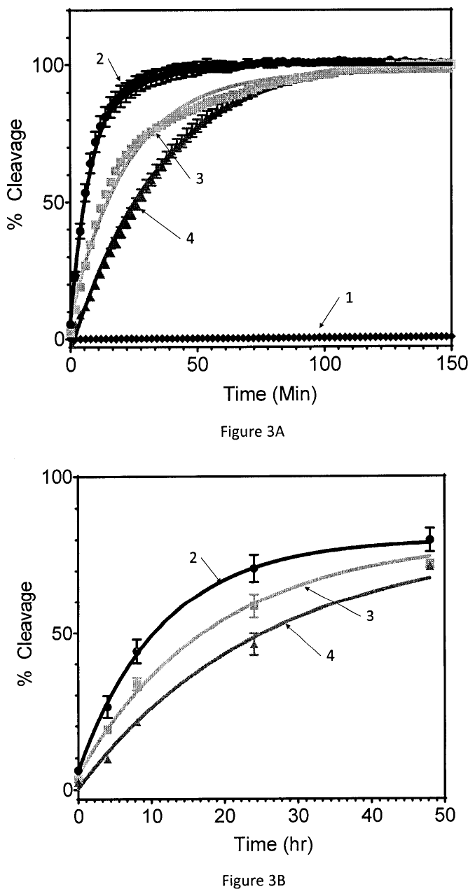

The in vitro cleavage of the compounds were assessed to determine their relative stabilities (FIG. 3A). The fluorescence of the compounds in buffered solution in the presence of dithiothreitol was monitored over a period of 2.5 hours. Compound 1 did not exhibit any increases in fluorescence, as expected from the inclusion of an uncleavable linker in this conjugate. Compound 4, which included a di-substituted carbon proximal to the disulfide, exhibited the slowest cleavage kinetics. Compounds 2 and 3, bearing unsubstituted and mono-substituted carbons next to the disulfide, respectively, exhibited faster cleavage kinetics, with compound 2 being cleaved with 10 minutes and compound 3 requiring approximately 45 minutes.

When fluorescence recovery correlated with linker cleavage was monitored in cellulo, a similar trend was observed (FIG. 3B). Cells were incubated with the compounds and the fluorescence of cell lysates was monitored over 48 hours. Compound 2 exhibited the fastest fluorescence recovery kinetics with saturation being reached in about 20 hours. However, this compound also exhibited measurable levels (10%) of cleavage directly following treatment, possibly suggesting cytosolic cleavage making it less attractive for mitochondrial delivery applications. The mono- and di-substituted conjugates exhibited lower levels of initial cleavage, and reached saturation over ˜48 hours.

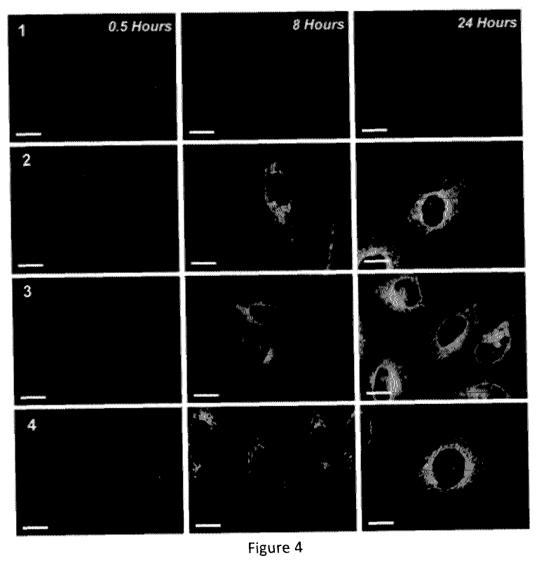

The time-dependence of linker cleavage was also confirmed visually using fluorescence microscopy. In experiments where all imaging conditions were held constant over the time course, all three disulfide-containing reporters exhibited a time dependent increase in fluorescence over time as opposed to the uncleavable control (FIG. 4). These results indicate that all three of these disulfides can be used for mitochondrial delivery, depending on the desired cleavage kinetics. The mono-substituted linker was prioritized as a platform for further development as it had low pre-incubation cleavage while still releasing the majority of its cargo within 24 hours.

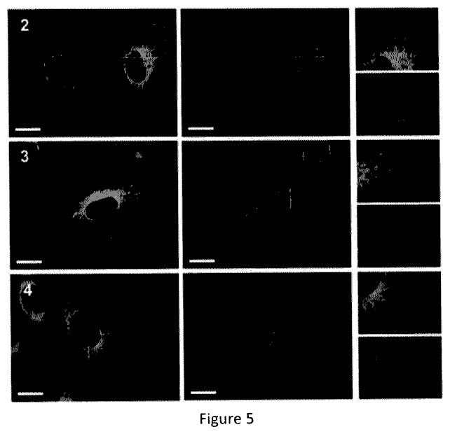

The extent of mitochondrial localization for the three disulfide linked compounds was also assessed (FIG. 5). When compared to a known mitochondrial stain (mitotracker deep red), all three of the disulfide-linked compounds exhibited high levels of co-localization. The extent of co-localization was assessed quantitatively through the calculation of Pearson's correlation coefficients, and the values were above 0.75 for all three conjugates.

### Example 2

To showcase the ability of this linker chemistry to release cargo into the mitochondrial matrix, the HSP90 inhibitor luminespib was used as a test cargo. HSP90 inhibitors have attracted intense pharmacological interest due to their chemotherapeutic properties and their lack of toxicity to non-cancer cells (see, e.g. reference 18, listed below). A number of HSP90 inhibitors have been developed in recent years targeting the cytoplasmic HSP90 pools of cancer cells. Inhibition of cytoplasmic HSP90 has been previously shown to cause arrest of cancer cell growth by antagonizing the stabilizing effect of HSP90 on signaling proteins involved in cancer cell growth and survival (see, e.g. reference 19, listed below). However, induction of cell death by cytoplasmic HSP90 inhibition has been found to be inconsistent, with some compounds inducing cell death in some cell lines and growth arrest in others (see, e.g. reference 20, listed below). This has led to difficulties in the clinical application of HSP90 inhibitors, especially as single agents (see, e.g. reference 21, listed below). Recent studies exploring HSP90 inhibitors delivered to the mitochondrial matrix via cationic vectors have suggested that inhibition of mitochondrial HSP90 and TRAP-1, a mitochondrial analogue, can more consistently and rapidly induce cell death via induction of apoptosis (see, e.g. references 22 and 23, listed below). However, IC50 values for the best characterized mitochondrial HSP90 inhibitors are relatively high (˜10 μM), indicating that cationic vectors may not lead to optimal efficacy.

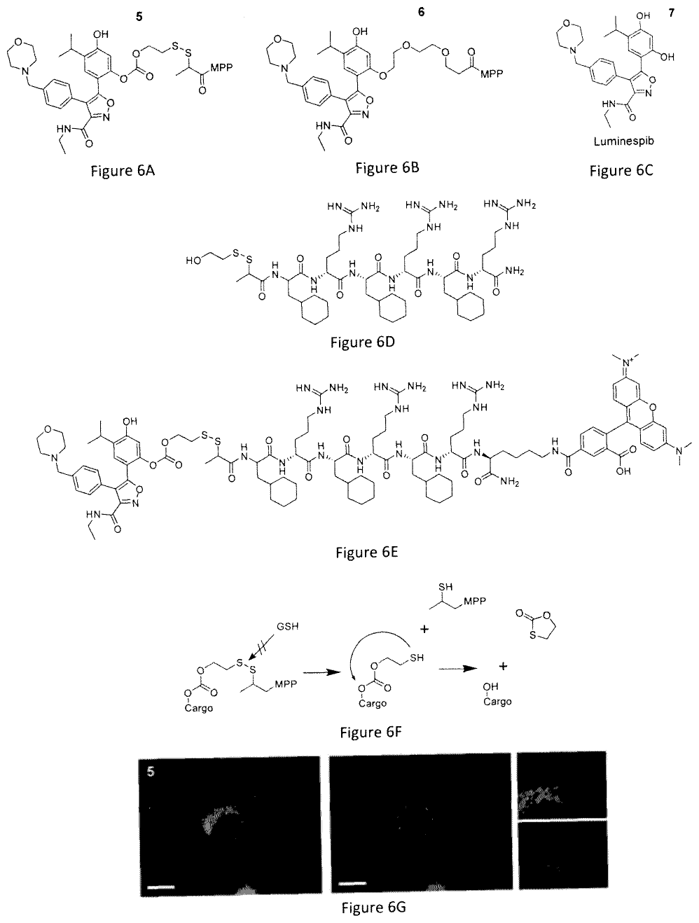

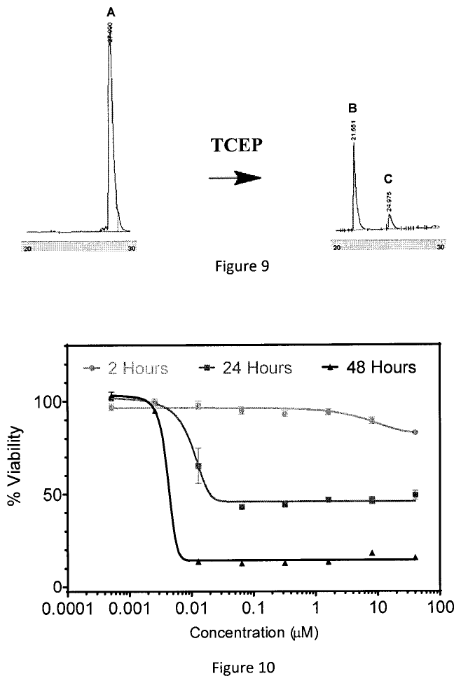

The HSP90 inhibitor luminespib was chosen as a candidate for the traceless linker approach. This compound has not previously been tested for mitochondrial activity because the functional groups that could be used for conjugation of a delivery vector are also involved directly in protein binding (see, e.g. reference 24, listed below). Luminespib was conjugated to a mitochondria-penetrating peptide via a mono-substituted disulfide as shown in FIG. 6A (compound 5). A non-cleavable analogue (compound 6) was also generated as shown in FIG. 6B. The structure of native luminespib is seen in FIG. 6C. The structure of the linker with the mitochondrial targeting vector is shown in FIG. 6D. The chemical structure of the fluorescently labelled analogue of compound 5 is shown in FIG. 6E. Cleavage of the disulfide linker in compound 5 by glutathione in the mitochondrial matrix was designed to trigger the release and regeneration of Luminespib through self-immolation of the thiol-carbonate (FIG. 6F). The regeneration of Luminespib after linker cleavage was shown to occur rapidly (FIG. 9), and mitochondrial localization of a fluorescently-labeled analogue was confirmed (FIG. 6G).

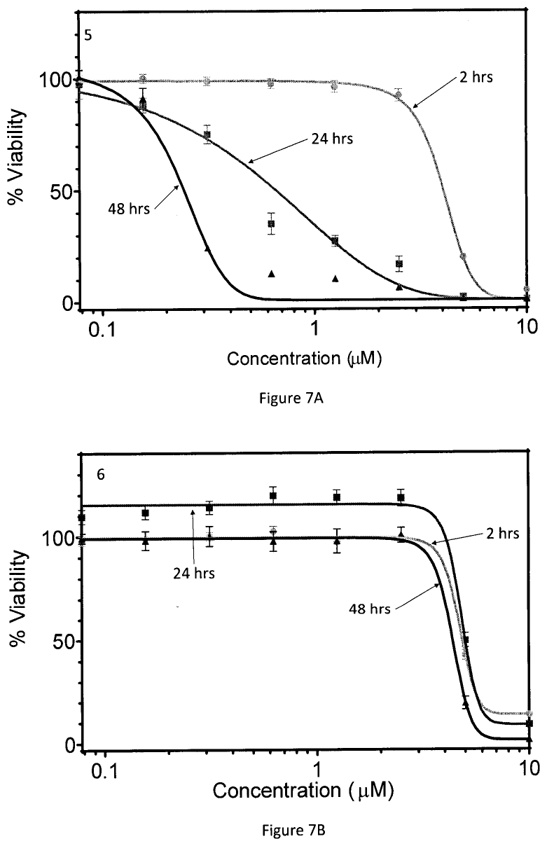

Leukemia cells treated with compound 5 exhibited a time dependent increase in cell toxicity over 48 hours (FIG. 7A) which was distinct from the growth inhibition induced by luminespib alone (FIG. 10). In contrast, the uncleavable compound 6 exhibited low levels of toxicity that remained static over time (FIG. 7B). At two hours, only a small amount of Luminespib would have been generated from compound 5, and the similarity between the toxicity between the two peptides suggests that the effects observed are from nonspecific toxicity of the peptides themselves, rather than an effect from the released Luminespib. Conversely, the time-dependent toxicity observed only with compound 5, and not the uncleavable compound 6, suggests that the difference in effects between the peptides is due to the cleavage and regeneration of Luminespib.

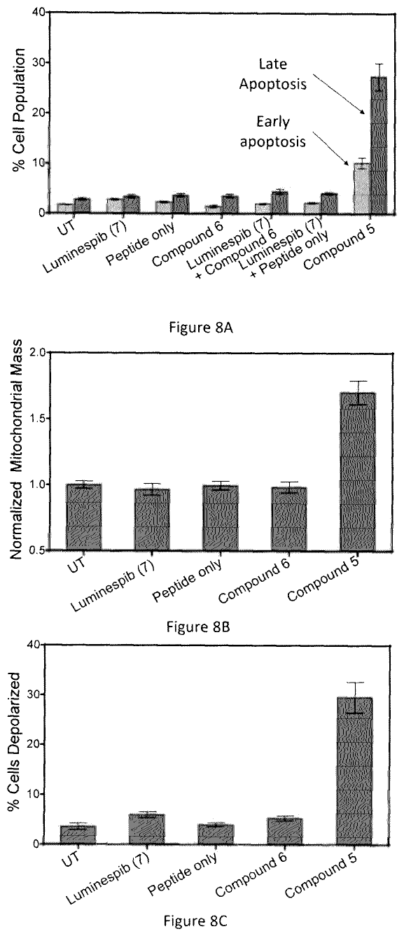

In order establish that the mechanism of cytotoxicity of mitochondrially-targeted luminespib (compound 5) was linked to mitochondrial effects, the mode of cell death was monitored (FIG. 8A), effects on mitochondrial mass (FIG. 8B), and mitochondrial depolarization (FIG. 8C). As controls, the parent compound luminespib (FIG. 8C, compound 7), and the empty disulfide vector (FIG. 6D) were also tested. The cells were treated with 2.5 μM of each compound. Compound 5 produced significant populations of early and late apoptotic cells after 24 hours as visualized by annexin V staining, as opposed to the parent compound and the peptide controls which exhibited no increase when tested (FIG. 8A). In addition, cotreatment of the parent compound with either the uncleavable compound 6 or the empty vector did not induce apoptosis, indicating that the effects observed with mitochondrially targeted Luminespib were not due to a nonspecific synergistic effect between the peptide and cytosolic HSP90 inhibition by luminespib. The mitochondrial mass of cells treated with the mitochondrially-targeted luminespib (compound 5) exclusively exhibited an increase in mitochondrial mass at 24 hours (FIG. 8B). These results suggest a cleavage specific induction of mitochondrial swelling, an indicator of mitochondrial toxicity and mitochondrial dependent apoptosis (see, e.g. reference 25, listed below). Cells treated with mitochondrially-targeted luminespib (compound 5) also exhibited mitochondrial depolarization, suggesting compromised mitochondrial integrity (FIG. 8C). In both experiments, no induction of mitochondrial dysfunction was observed in any of the control compounds, suggesting the effects induced by mitochondrially-targeted luminespib (compound 5) derived free luminespib generated in the mitochondrial matrix and not from the vector itself.

### Example 3

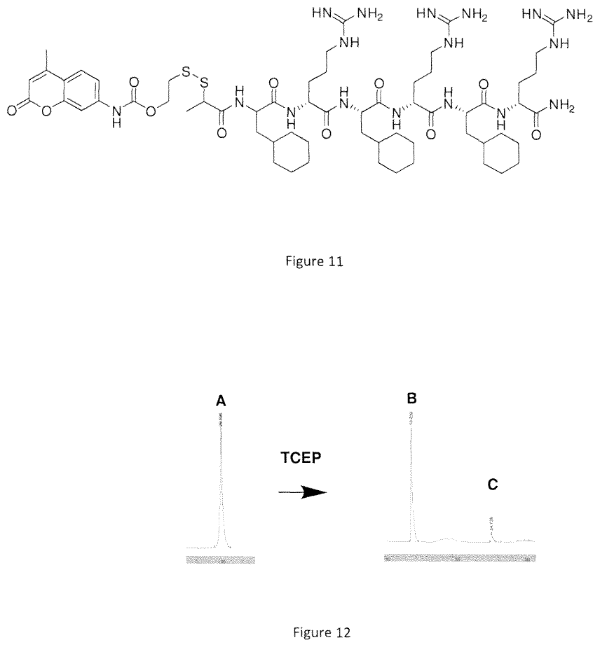

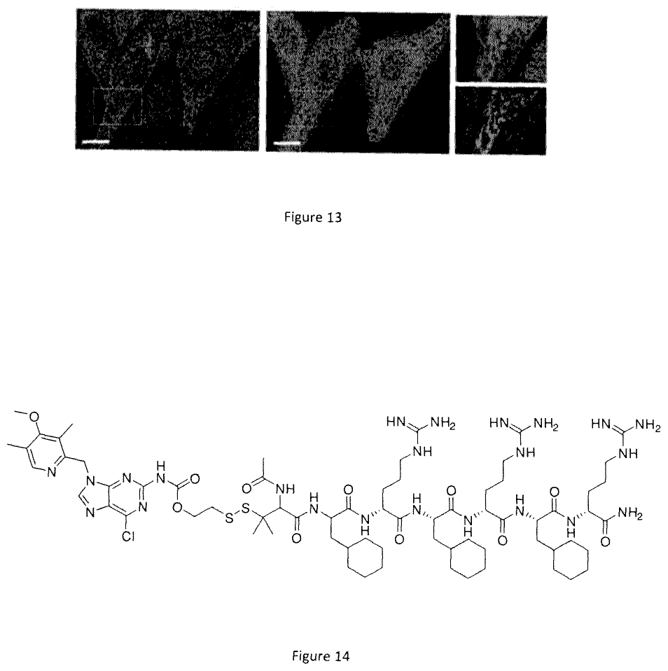

In order to establish the ability of the linker system to be used with compounds containing amine groups, a mitochondrially targeted 7-amino-4-methylcoumarin conjugate was synthesized using the releasable linker (FIG. 11). The extent of mitochondrial localization of the releasable aminocoumarin peptide was assessed (FIG. 12). When compared to a known mitochondrial stain (mitotracker deep red), the compound exhibited high levels of co-localization. The extent of co-localization was assessed quantitatively through the calculation of Pearson's correlation coefficient, and the values was found to be 0.73 for the conjugate. The regeneration of 7-amino-4-methylcoumarin after linker cleavage was shown to occur within 4 hours of linker cleavage (FIG. 13).

### Example 4

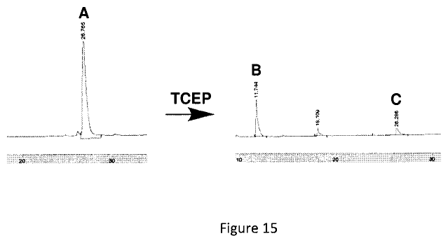

A mitochondrially targeted BIIB021 conjugate was synthesized using the releasable linker (FIG. 14). The regeneration of BIIB021 after linker cleavage was shown to occur within 4 hours of linker cleavage (FIG. 15).

These results show a system for chemical cargo release from mitochondria-targeting vectors using a flexible and enzyme-independent platform. This strategy may be used to localize compounds to the mitochondria which have functional groups that otherwise make them incompatible with targeting vectors. The results also show that the kinetics of the chemical cleavage of disulfide linkers in the mitochondria differ than what would be expected from in vitro data, and outline a reporter system that can be used to determine linker stability in the mitochondria.

Although preferred embodiments of the invention have been described herein, it will be understood by those skilled in the art that variations may be made thereto without departing from the spirit of the invention or the scope of the appended claims. All references disclosed herein are incorporated in the entirety by reference.

### REFERENCES

1. Nunnari, J.; Suomalainen, A., Mitochondria: in sickness and in health. Cell 2012, 148 (6), 1145-59.

2. Smith, R. A.; Hartley, R. C.; Murphy, M. P., Mitochondria-targeted small molecule therapeutics and probes. Antioxid Redox Signal 2011, 15 (12), 3021-38.

3. Horton, K. L.; Stewart, K. M.; Fonseca, S. B.; Guo, Q.; Kelley, S. O., Mitochondria-penetrating peptides. Chem Biol 2008, 15 (4), 375-82.

4. Jean, S. R.; Ahmed, M.; Lei, E. K.; Wisnovsky, S. P.; Kelley, S. O., Peptide-Mediated Delivery of Chemical Probes and Therapeutics to Mitochondria. Acc Chem Res 2016, 49 (9), 1893-902.

5. Wisnovsky, S.; Lei, E. K.; Jean, S. R.; Kelley, S. O., Mitochondrial Chemical Biology: New Probes Elucidate the Secrets of the Powerhouse of the Cell. Cell Chem Biol 2016, 23 (8), 917-27.

6. Fonseca, S. B.; Pereira, M. P.; Mourtada, R.; Gronda, M.; Horton, K. L.; Hurren, R.; Minden, M. D.; Schimmer, A. D.; Kelley, S. O., Rerouting chlorambucil to mitochondria combats drug deactivation and resistance in cancer cells. Chem Biol 2011, 18 (4), 445-53.

7. Jean, S. R.; Tulumello, D. V.; Riganti, C.; Liyanage, S. U.; Schimmer, A. D.; Kelley, S. O., Mitochondrial Targeting of Doxorubicin Eliminates Nuclear Effects Associated with Cardiotoxicity. ACS Chem Biol 2015, 10 (9), 2007-15.

8. Wisnovsky, S.; Jean, S. R.; Kelley, S. O., Mitochondrial DNA repair and replication proteins revealed by targeted chemical probes. Nat Chem Biol 2016, 12 (7), 567-73.

9. Chamberlain, G. R.; Tulumello, D. V.; Kelley, S. O., Targeted delivery of doxorubicin to mitochondria. ACS Chem Biol 2013, 8 (7), 1389-95.

10. Wisnovsky, S. P.; Wilson, J. J.; Radford, R. J.; Pereira, M. P.; Chan, M. R.; Laposa, R. R.; Lippard, S. J.; Kelley, S. O., Targeting mitochondrial DNA with a platinum-based anticancer agent. Chem Biol 2013, 20 (11), 1323-8.

11. Pathak, R. K.; Marrache, S.; Harn, D. A.; Dhar, S., Mito-DCA: a mitochondria targeted molecular scaffold for efficacious delivery of metabolic modulator dichloroacetate. ACS Chem Biol 2014, 9 (5), 1178-87.

12. Ripcke, J.; Zarse, K.; Ristow, M.; Birringer, M., Small-molecule targeting of the mitochondrial compartment with an endogenously cleaved reversible tag. Chembiochem 2009, 10 (10), 1689-96.

13. Brülisauer, L.; Gauthier, M. A.; Leroux, J. C., Disulfide-containing parenteral delivery systems and their redox-biological fate. J Control Release 2014, 195, 147-54.

14. Gasparini, G.; Matile, S., Protein delivery with cell-penetrating poly(disulfide)s. Chem Commun (Camb) 2015, 51 (96), 17160-2.

15. Brezden, A.; Mohamed, M. F.; Nepal, M.; Harwood, J. S.; Kuriakose, J.; Seleem, M. N.; Chmielewski, J., Dual Targeting of Intracellular Pathogenic Bacteria with a Cleavable Conjugate of Kanamycin and an Antibacterial Cell-Penetrating Peptide. J Am Chem Soc 2016, 138 (34), 10945-9.

16. Marí, M.; Morales, A.; Colell, A.; García-Ruiz, C.; Fernández-Checa, J. C., Mitochondrial glutathione, a key survival antioxidant. Antioxid Redox Signal 2009, 11 (11), 2685-700.

17. Lewis Phillips, G. D.; Li, G.; Dugger, D. L.; Crocker, L. M.; Parsons, K. L.; Mai, E.; Blather, W. A.; Lambert, J. M.; Chari, R. V.; Lutz, R. J.; Wong, W. L.; Jacobson, F. S.; Koeppen, H.; Schwall, R. H.; Kenkare-Mitra, S. R.; Spencer, S. D.; Sliwkowski, M. X., Targeting HER2-positive breast cancer with trastuzumab-DM1, an antibody-cytotoxic drug conjugate. Cancer Res 2008, 68 (22), 9280-90.

18. Butler, L. M.; Ferraldeschi, R.; Armstrong, H. K.; Centenera, M. M.; Workman, P., Maximizing the Therapeutic Potential of HSP90 Inhibitors. Mol Cancer Res 2015, 13 (11), 1445-51.

19. Trepel, J.; Mollapour, M.; Giaccone, G.; Neckers, L., Targeting the dynamic HSP90 complex in cancer. Nat Rev Cancer 2010, 10 (8), 537-49.

20. Neckers, L., Hsp90 inhibitors as novel cancer chemotherapeutic agents. Trends Mol Med 2002, 8 (4 Suppl), S55-61.

21. Neckers, L.; Workman, P., Hsp90 molecular chaperone inhibitors: are we there yet? Clin Cancer Res 2012, 18 (1), 64-76.

22. Lee, C.; Park, H. K.; Jeong, H.; Lim, J.; Lee, A. J.; Cheon, K. Y.; Kim, C. S.; Thomas, A. P.; Bae, B.; Kim, N. D.; Kim, S. H.; Suh, P. G.; Ryu, J. H.; Kang, B. H., Development of a mitochondria-targeted Hsp90 inhibitor based on the crystal structures of human TRAP1. J Am Chem Soc 2015, 137 (13), 4358-67.

23. Kang, B. H.; Plescia, J.; Song, H. Y.; Meli, M.; Colombo, G.; Beebe, K.; Scroggins, B.; Neckers, L.; Altieri, D. C., Combinatorial drug design targeting multiple cancer signaling networks controlled by mitochondrial Hsp90. J Clin Invest 2009, 119 (3), 454-64.

24. Brough, P. A.; Aherne, W.; Barril, X.; Borgognoni, J.; Boxall, K.; Cansfield, J. E.; Cheung, K. M.; Collins, I.; Davies, N. G.; Drysdale, M. J.; Dymock, B.; Eccles, S. A.; Finch, H.; Fink, A.; Hayes, A.; Howes, R.; Hubbard, R. E.; James, K.; Jordan, A. M.; Lockie, A.; Martins, V.; Massey, A.; Matthews, T. P.; McDonald, E.; Northfield, C. J.; Pearl, L. H.; Prodromou, C.; Ray, S.; Raynaud, F. I.; Roughley, S. D.; Sharp, S. Y.; Surgenor, A.; Walmsley, D. L.; Webb, P.; Wood, M.; Workman, P.; Wright, L., 4,5-diarylisoxazole Hsp90 chaperone inhibitors: potential therapeutic agents for the treatment of cancer. J Med Chem 2008, 51 (2), 196-218.

25. Desagher, S.; Martinou, J. C., Mitochondria as the central control point of apoptosis. Trends Cell Biol 2000, 10 (9), 369-77.

1. A compound comprising:
a mitochondrial targeting portion;
a cargo portion including a drug unit; and
a linker conjugating the mitochondrial targeting portion and the cargo portion, the linker portion cleavable in a mitochondrion of a cell for preferentially releasing the cargo portion within the mitochondrion as compared to a cytoplasm of the cell.

2. The compound of claim 1, wherein the linker portion comprises disulfide.

3. The compound of claim 2, wherein each carbon atom bonded to the disulfide is, independently, unsubstituted; mono- or di-substituted by, independently, a hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl group; or di-substituted such that the carbon atom bonded to the disulfide forms part of a C₃-C₈ cycloalkyl, or C₃-C₈ cycloalkenyl.

4. The compound of claim 1, wherein the drug unit includes a hydroxyl, amine or thiol group.

5. The compound of claim 1, wherein the cargo portion includes an auto-cyclization moiety that activates by the cleavage of the linker to release the drug unit.

6. The compound of claim 5, wherein the auto-cyclization moiety includes an ester moiety that reacts with a moiety of the cleaved linker portion.

7. The compound of claim 6, wherein the moiety of the cleaved linker portion that reacts with the ester moiety is a sulfur moiety.

8. The compound of claim 6, wherein the drug unit includes an oxygen moiety bonded to the ester moiety to form a carbonate moiety, wherein the auto-cyclization cleaves the cargo unit portion at the oxygen-carbon bond of the carbonate moiety such that the oxygen moiety of the released drug unit forms a hydroxyl group.

9. The compound of claim 6, wherein the drug unit includes an nitrogen moiety bonded to the ester moiety to form a carbamate moiety, wherein the auto-cyclization cleaves the cargo unit portion at the nitrogen-carbon bond of the carbonate moiety such that the nitrogen moiety of the released drug unit forms an amine group.

10-34. (canceled)

35. A compound having a structure according to Formula I: 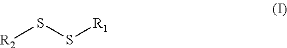
wherein R₁ is a mitochondrial targeting portion;
R₂ is a cargo portion including a drug unit; and
each carbon atom bonded to the disulfide is, independently, unsubstituted; mono- or di-substituted by, independently, a hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl group; or di-substituted such that the carbon atom bonded to the disulfide forms part of a C₃-C₈ cycloalkyl, or C₃-C₈ cycloalkenyl group.

36. The compound of claim 35 wherein the mitochondrial targeting portion includes a mitochondrial penetrating peptide (MPP), a triphenylphosphonium (TPP), a transactivator of transcription peptide fused mitochondrial targeting sequence (TAT-MTS), a mitochondrial protein, or a small molecule with mitochondrial localization.

37. The compound of claim 35 wherein R₁ has a structure according to Formula II: 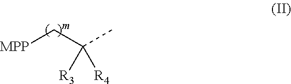
wherein R₃ and R₄ are, independently, hydrogen, hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl; or R₃ and R₄ together form C₃-C₈ cycloalkyl, or C₃-C₈ cycloalkenyl; and m is an integer from 0 to 8.

38. The compound of claim 37 wherein the MPP has a structure according to Formula IIa:

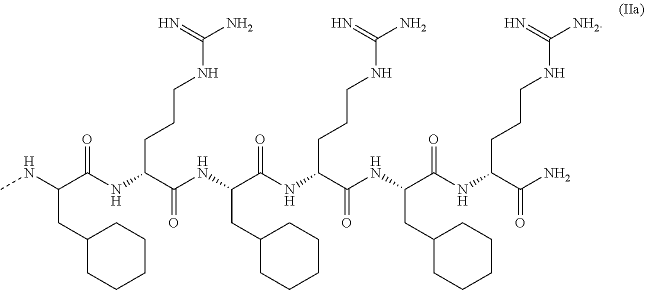

39. The compound of claim 35, wherein R₂ has a structure according to Formula III: 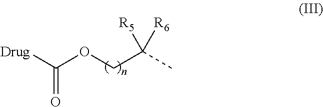
wherein R₅ and R₆ are, independently, hydrogen, hydroxyl, amino, fluoro, chloro, bromo, C₁-C₄ alkyl, C₁-C₄ alkenyl, C₁-C₄ alkynyl, C₃-C₈ cycloalkyl, C₃-C₈ cycloalkenyl, or phenyl; or R₅ and R₆ together form C₃-C₈ cycloalkyl, or C₃-C₈ cycloalkenyl;
n is an integer from 1 to 4; and
Drug is the drug unit.

40. The compound of claim 35, wherein the drug unit includes a heat shock protein 90 (HSP90) inhibitor, pyruvate dehydrogenase kinase modulator, SIRT1 modulator, mitochondrial estrogen receptor ligand, mtDNA synthesis modulator, modulator of mtDNA fidelity, mitochondrial pol theta modulator, cyclophilin modulator, mitochondrial metabolism modulator, hexokinase modulator, lactate dehydrogenase modulator, glucose-6-phosphate modulator, kynurenine 3-monooxygenease modulator, AMP-activated protein kinase modulator, POLRMT modulator, or PINK1 modulator.

41. The compound of claim 40, wherein the HSP90 inhibitor is luminespib, ganetespib, onalespib, SNX-2112, SNX-5422, KW2478, NMS-E973, VER-49009, or VER-50589.

42. The compound of claim 41, wherein the HSP90 inhibitor is luminespib.

43. The compound of claim 35, wherein the drug unit includes a small molecule drug.

44. The compound of claim 35, wherein the drug unit includes a peptide.

45. The compound of claim 44, wherein the peptide has from 3-mer to 30-mer units.

46-48. (canceled)


SEQUENCE LISTING (10 sequences)

SEQ ID NO: 1 - protein, 4 aa, Artificial Sequence
  Synthetic peptide
  MISC_FEATURE (1)..(1): Xaa is cyclohexamide
  MISC_FEATURE (2)..(2): Xaa is D-arginine
  MISC_FEATURE (3)..(3): Xaa is cyclohexamide
XXXK

SEQ ID NO: 2 - protein, 8 aa, Artificial Sequence
  Synthetic Peptide
  MISC_FEATURE (1)..(1): Xaa is cyclohexylalanine
  MISC_FEATURE (2)..(2): Xaa is D-arginine
  MISC_FEATURE (3)..(3): Xaa is cyclohexylalanine
  MISC_FEATURE (5)..(5): Xaa is cyclohexylalanine
  MISC_FEATURE (6)..(6): Xaa is D-arginine
  MISC_FEATURE (7)..(7): Xaa is cyclohexylalanine
XXXKXXXK

SEQ ID NO: 3 - protein, 8 aa, Artificial Sequence
  Synthetic Peptide
  MISC_FEATURE (2)..(2): Xaa is D-arginine
  MISC_FEATURE (3)..(3): Xaa is cyclohexylalanine
  MISC_FEATURE (6)..(6): Xaa is D-arginine
  MISC_FEATURE (7)..(7): Xaa is cyclohexylalanine
FXXKFXXK

SEQ ID NO: 4 - protein, 4 aa, Artificial Sequence
  Synthetic peptide
  MISC_FEATURE (2)..(2): Xaa is D-arginine
  MISC_FEATURE (3)..(3): Xaa is cyclohexylalanine
FXXK

SEQ ID NO: 5 - protein, 4 aa, Artificial Sequence
  Synthetic peptide
  MISC_FEATURE (2)..(2): Xaa is D-arginine
  MISC_FEATURE (3)..(3): Xaa is diphenylalanine
FXXK

SEQ ID NO: 6 - protein, 4 aa, Artificial Sequence
  Synthetic peptide
  MISC_FEATURE (2)..(2): Xaa is D-arginine
  MISC_FEATURE (3)..(3): Xaa is (1-naphthyl)-L-alanine
FXXK

SEQ ID NO: 7 - protein, 4 aa, Artificial Sequence
  Synthetic peptide
  MISC_FEATURE (2)..(2): Xaa is D-arginine
  MISC_FEATURE (3)..(3): Xaa is 2-aminooctanoic acid
FXXK

SEQ ID NO: 8 - protein, 8 aa, Artificial Sequence
  Synthetic peptide
  MISC_FEATURE (1)..(1): Xaa is cyclohexylalanine
  MISC_FEATURE (2)..(2): Xaa is D-arginine
  MISC_FEATURE (3)..(3): Xaa is cyclohexylalanine
  misc_feature (4)..(4): Xaa can be any naturally occurring amino acid
  MISC_FEATURE (5)..(5): Xaa is cyclohexylalanine
  MISC_FEATURE (6)..(6): Xaa is D-arginine
  MISC_FEATURE (7)..(7): Xaa is cyclohexylalanine
  MISC_FEATURE (8)..(8): Xaa is D-arginine
XXXXXXXX

SEQ ID NO: 9 - protein, 12 aa, Artificial Sequence
  Synthetic peptide
  MISC_FEATURE (1)..(1): Xaa is cyclohexylalanine
  MISC_FEATURE (2)..(2): Xaa is D-arginine
  MISC_FEATURE (4)..(4): Xaa is D-arginine
  MISC_FEATURE (5)..(5): Xaa is cyclohexylalanine
  MISC_FEATURE (6)..(6): Xaa is D-arginine
  MISC_FEATURE (8)..(8): Xaa is D-arginine
  MISC_FEATURE (9)..(9): Xaa is cyclohexylalanine
  MISC_FEATURE (10)..(10): Xaa is D-arginine
  MISC_FEATURE (12)..(12): Xaa is D-arginine
XXGXXXGXXXGX

SEQ ID NO: 10 - protein, 6 aa, Artificial Sequence
  Synthetic peptide
  MISC_FEATURE (1)..(1): Xaa is cyclohexylalanine
  MISC_FEATURE (2)..(2): Xaa is D-arginine
  MISC_FEATURE (3)..(3): Xaa is cyclohexylalanine
  MISC_FEATURE (4)..(4): Xaa is D-arginine
  MISC_FEATURE (5)..(5): Xaa is cyclohexylalanine
  MISC_FEATURE (6)..(6): Xaa is D-arginine
XXXXXX